(19) United States
(12) Patent Application Publication
(10) Pub. No.: US 2022/0409884 A1
(43) Pub. Date: Dec. 29, 2022

(54) SUBCUTANEOUS IMPLANTABLE DEFIBRILLATOR WITH EPICARDIAL LEAD FOR RESYNCHRONIZATION THERAPY

(71) Applicant: NewPace Ltd., Caesarea (IL)

(72) Inventors: Gera Strommer, Caesarea (IL); Avraham Broder, Caesarea (IL); Robert Fishel, Caesarea (IL)

(21) Appl. No.: 17/897,480

(22) Filed: Aug. 29, 2022

Related U.S. Application Data
(63) Continuation of application No. 16/611,405, filed on Nov. 6, 2019.
(60) Provisional application No. 62/502,687, filed on May 7, 2017.

(51) Int. Cl. A61N 1/05; A61N 1/39; A61N 1/365; A61N 1/378

(52) U.S. Cl. CPC A61N 1/0504; A61N 1/39622; A61N 1/059; A61N 1/365; A61N 1/3787

(57) ABSTRACT

Subcutaneous implantable string shaped defibrillator for providing cardiac resynchronization therapy (CRT), including a flexible elongated body, at least two defibrillation leads, at least one sensor, at least two transition units and at least one epicardial lead, the defibrillation leads for providing at least one cardioversion defibrillation shock, the sensor being positioned on at least one of the defibrillation leads, for determining at least one metric of a heart, the transition units for respectively coupling the defibrillation leads to opposite ends of the elongated body, and the epicardial lead, coupled with the elongated body via at least one of the transition units, for providing at least one CRT pulse, the elongated body including a plurality of linked units, the linked units encapsulating at least one capacitor, at least one power source and a processor, wherein the processor provides at least one signal to the epicardial lead for providing the CRT pulse.

### CROSS REFERENCE TO RELATED APPLICATIONS

This is a continuation of U.S. patent application Ser. No. 16/611,405 filed Nov. 6, 2019, which was a 371 application from international patent application No. PCT/IL2018/050473 filed Apr. 30, 2018, which claims the benefit of priority from U.S. Provisional patent applications No. 62/502,687 filed May 7, 2017.

### FIELD OF THE DISCLOSED TECHNIQUE

The disclosed technique relates to subcutaneous implantable cardioversion defibrillators and pacemakers, in general, and to methods and systems for applying resynchronization therapy via an epicardial lead from a subcutaneous implantable pulse generator, in particular.

### BACKGROUND OF THE DISCLOSED TECHNIQUE

Implantable cardioversion defibrillators (herein abbreviated ICDs), pacemakers and cardiac resynchronization therapy (herein abbreviated CRT) devices are known in the art. All these devices are used to treat patients with various types of heart arrhythmias. ICDs in particular are used to terminate ventricular fibrillation (herein abbreviated VF) and ventricular tachycardia (herein abbreviated VT) which can lead to sudden cardiac arrest (herein abbreviated SCA). CRT devices are used to pace the heart as well as to resynchronize the beating of the ventricles of the heart such that they work in a coordinated manner. CRT devices with the ability to defibrillate as well (similar to ICDs) are referred to as CRT-D devices. Many ICDs and CRT-D devices consist of an implantable pulse generator (herein abbreviated IPG) in the form of a can along with leads (also known as a can and leads design) wherein the IPG, housing electronics, at least one capacitor and a battery, is implanted subcutaneously and the leads are implanted intravascularly in the heart. Depending on the heart condition of a patient, the leads may be placed in the right atrium, the right ventricle, directed towards the left atrium and/or in the left ventricle of the heart of the patient, mostly through the coronary sinus vein which is external to the left ventricle and may be accessed via the coronary sinus ostium located in the right atrium. Such devices usually require major surgery to properly position the leads in the heart and may operate for between 5-7 years before the battery in the IPG needs to be replaced via another surgery.

The functions performed by a transvenous ICD (where the leads are implanted intravascularly) can be performed by a subcutaneous ICD having both an IPG and leads which are positioned subcutaneously, i.e. a fully subcutaneous ICD with no leads in the heart. An example of such a device is the EMBLEM MRI S-ICD™ System manufactured by Boston Scientific® which includes an IPG positioned just outside the ribcage and a lead positioned subcutaneously on top of the sternum. A subcutaneous ICD can detect arrhythmias such as VF and VT, which may lead to SCA, with no sensors placed directly in or on the heart and can apply high voltage electric shocks to restore the heart to a normal rhythm. In the case of a CRT however, performing the function of pacing subcutaneously is more of a challenge. Firstly, low voltage electric shocks need to be delivered constantly without interfering with normal heart function. Secondly, it is difficult to detect when the right and/or left ventricles are to be paced without a sensor placed directly in the ventricles to detect when they are naturally contracting to thus determine when they should be contracting.

For patients who require resynchronization therapy due to left bundle branch block (herein abbreviated BBB) and who are also at risk for SCA, transvenous CRT-D devices are used. The defibrillation function is provided by the IPG and two leads placed in the right atrium and right ventricle to prevent SCA. Resynchronization therapy pulses for pacing the left ventricle due to left BBB are delivered to the left side of the heart through a third lead placed within the coronary sinus. As mentioned above, a subcutaneous ICD has the advantage of not having any leads within the heart and/or vascular system however such a device cannot deliver resynchronization therapy pulses and therefore may be used in only a limited portion of the target population for heart devices. For patients who have a blocked coronary sinus or via which the left side of the heart is inaccessible intravascularly, some CRT-D devices have an IPG outfitted with an additional lead for epicardial placement on the left side of the heart which can be used to treat patients suffering from left BBB. The additional lead is attached externally to the heart and is implanted via surgical access to the IPG and then tunneled under the skin from the IPG location to the area of the apex of the heart. The additional lead usually has a screw end which forms part of the lead. A tiny surgical incision is then made below the ribs where the screw end is affixed to the outer surface of the heart muscle. Such epicardial leads are connected to the IPG using ISO standard 5841-3:2013 (also known as IS-1 connectors) which specifies a connector assembly to connect implantable pacemaker leads to implantable pacemaker pulse generators as well as to CRT-D devices. Many ICD companies, such as Medtronic®, Abbott® and Boston Scientific®, manufacture such off-the-shelf leads.

It is noted that some ICD companies, such as Boston Scientific® and ST. JUDE MEDICALTM are trying to resolve the issue of a subcutaneous ICD not being able to provide CRT-D by using a pill pacemaker located within the right ventricle and implanted percutaneously which can communicate wirelessly with a subcutaneous ICD. Such pill pacemaker devices are known in the art, such as the MICRA™ by Medtronic® and the NANOSTIM™ by ST. JUDE MEDICAL™. The disadvantage of such a solution is that the pill pacemaker must still be implanted within the heart and must continuously communicate with the subcutaneous ICD, thus shortening the lifespan of the battery of the subcutaneous ICD.

Reference is now made to FIG. 1, which is a schematic illustration of a CRT-D device with an epicardial lead, generally referenced 10, as is known in the prior art. FIG. 1 shows a heart 12 in which an intravascular ICD 14 has been implanted. Intravascular ICD 14 includes an IPG 15 and a plurality of leads 24A, 24B and 32. IPG 15 includes a main portion 26 and a connector 28. Main portion 26 may include a battery, electronics and at least one capacitor (all not shown). Connector 28 enables plurality of leads 24A, 24B and 32 to be coupled with main portion 26. As shown, heart 12 includes a right atrium 16, a right ventricle 20, a left atrium 20 and a left ventricle 22. Right atrium 16 and right ventricle 20 are shown in a cut-out 36 to show the placement of plurality of leads 24A and 24B within heart 12. As shown, lead 24A is placed intravascularly in right atrium 16 and is affixed to the inner wall 40 of right atrium 16. Lead 24B is placed intravascularly in right ventricle 18 and is affixed to the inner wall 42 of right ventricle 18. Lead 24B passes through the tricuspid valve, shown as two sections 38A and 38B. Plurality of leads 24A and 24B along with IPG 15 form a regular CRT-D or ICD. As shown, lead 32 is positioned epicardially on the outer surface of left ventricle 22, coupled via an epicardial connector 34. Lead 32 thus enables intravascular ICD 14 to function as a CRT device and to aid patients suffering from left BBB with a blocked coronary sinus wherein intravascular placement of a lead in the left side of the heart is not possible.

### SUMMARY OF THE DISCLOSED TECHNIQUE

The disclosed technique provides a novel system for a subcutaneous implantable heart device capable of applying defibrillation shocks, as well as resynchronization therapy via an epicardial lead, which overcomes the disadvantages of the prior art. According to an aspect of the disclosed technique, there is thus provided a subcutaneous implantable string shaped defibrillator (IS SD) for providing cardiac resynchronization therapy (CRT) (ISSD-T). The ISSD-T includes a flexible elongated body, at least two defibrillation leads, at least one sensor, at least two transition units and at least one epicardial lead. The sensor is positioned on at least one of the defibrillation leads and the epicardial lead is coupled with the flexible elongated body via at least one of the transition units. The defibrillation leads are for providing at least one cardioversion defibrillation shock. The sensor is for determining at least one metric of a heart. The transition units are for respectively coupling each one of the defibrillation leads to opposite ends of the flexible elongated body. The epicardial lead is for providing at least one CRT pulse. The flexible elongated body includes a plurality of linked units. At least a first one of the linked units encapsulates at least one capacitor, at least a second one of the linked units encapsulates at least one power source and at least a third one of the linked units encapsulates a processor. The processor provides at least one signal to the epicardial lead for providing the CRT pulse.

According to another aspect of the disclosed technique, there is thus provided a subcutaneous implantable defibrillator for providing cardiac resynchronization therapy (CRT). The subcutaneous implantable defibrillator includes an implantable pulse generator (IPG), at least one defibrillation lead and at least one epicardial lead. The defibrillation lead is positioned external to a heart. The epicardial lead is for providing at least one CRT pulse. The IPG includes a connector box, at least one capacitor, at least one power source and at least one electronic circuit. The connector box is for coupling the defibrillation lead and the epicardial lead with the IPG. The defibrillation lead includes at least one sensor for determining at least one metric of the heart. The epicardial lead provides the CRT pulse according to the determined metric.

### BRIEF DESCRIPTION OF THE DRAWINGS

The disclosed technique will be understood and appreciated more fully from the following detailed description taken in conjunction with the drawings in which:

FIG. 1 is a schematic illustration of a CRT-D device with an epicardial lead, as is known in the prior art;

FIG. 2 is a schematic illustration of a subcutaneous implantable string shaped defibrillator with an epicardial lead positioned in a patient, constructed and operative in accordance with an embodiment of the disclosed technique;

FIG. 3 is a schematic illustration of various epicardial lead ends for coupling the epicardial lead to a left ventricle of a heart, constructed and operative in accordance with another embodiment of the disclosed technique;

FIG. 4 is a schematic illustration of an epicardial lead end for coupling the epicardial lead to a subcutaneous implantable string shaped defibrillator, constructed and operative in accordance with a further embodiment of the disclosed technique;

FIG. 5 is a schematic illustration of variations of the subcutaneous implantable string shaped defibrillator with an epicardial lead of FIG. 2, constructed and operative in accordance with another embodiment of the disclosed technique;

FIG. 6 is a schematic illustration of a subcutaneous implantable string shaped defibrillator with two epicardial leads, constructed and operative in accordance with a further embodiment of the disclosed technique; and

FIG. 7 is a schematic illustration of a subcutaneous ICD with an epicardial lead, constructed and operative in accordance with another embodiment of the disclosed technique.

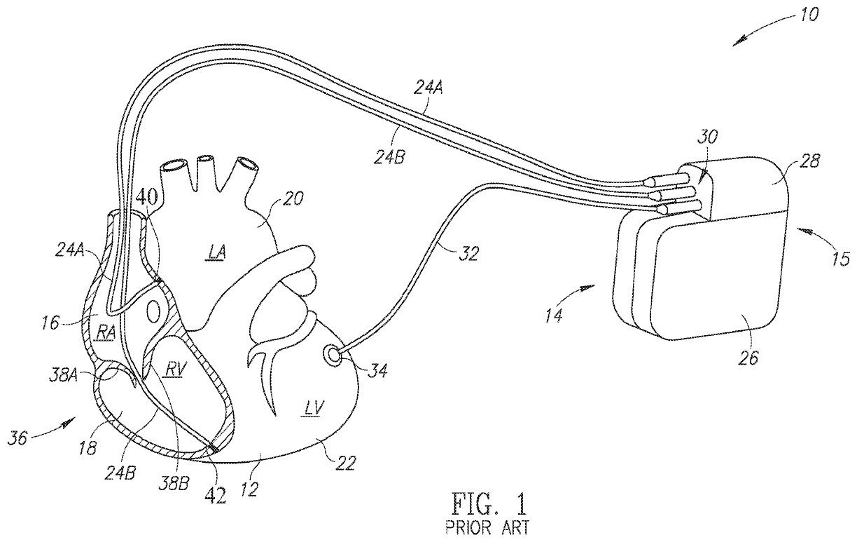

### DETAILED DESCRIPTION OF THE EMBODIMENTS

The disclosed technique overcomes the disadvantages of the prior art by providing a fully subcutaneous heart device with an epicardial lead thus enabling the functions of an ICD and a CRT to be achieved by the disclosed technique without any leads placed in the heart or in the vascular system. As described below, the disclosed technique is generally described using a subcutaneous implantable string shaped defibrillator (herein abbreviated ISSD) which is completely subcutaneous, is flexible and does not involve a separate IPG and leads design. However the disclosed technique can be embodied using any subcutaneous heart device which does not include any elements or components which are placed in the heart or the vascular system, such as a subcutaneous ICD having a subcutaneously placed IPG and a subcutaneously placed lead above or around the sternum (such as shown in U.S. Pat. Nos. 8,644,926 B2 and 6,721,597 B1) or a subcutaneous ICD having a subcutaneously placed IPG and a substernally placed lead (such as shown in US patent application publication no. 2014/0330327 A1). The subcutaneous ISSD is a single structure with no detachable parts and is placed subcutaneously around the heart for providing defibrillation shocks in the event of VF or VT. According to the disclosed technique, the described subcutaneous ISSD (or subcutaneous heart device in general) is designed for resynchronization therapy (herein abbreviated ISSD-T) as well and includes an epicardial lead which can be coupled epicardially to the outer surface of a heart. The IS SD-T utilizes an anticipative pacing algorithm and method for determining when to pace the left and/or right ventricles of the heart without requiring a lead to be placed in the heart to detect when the ventricles contract naturally and to determine when they should be paced. As mentioned above, the disclosed technique can be embodied by a subcutaneous ICD having an IPG with a subcutaneously or substernally positioned lead and also having an epicardial lead and using an anticipative pacing algorithm. According to the disclosed technique, a completely subcutaneous heart device (such as a subcutaneous ICD or a flexible subcutaneous ISSD) is provided which also allows for both defibrillation and cardiac resynchronization therapy to be applied to the heart. Defibrillation can be applied via subcutaneously or substernally placed leads and CRT can be applied via the epicardial lead. An anticipative pacing algorithm is used to determine when to pace the left and/or right ventricles of the heart thus obviating the need for any leads to be placed in the heart to determine when the ventricles are to be paced. Thus according to the disclosed technique, a completely subcutaneous heart device with no leads in the heart or vasculature can be used to pace the left side of the heart (for example, for patients suffering from left BBB), the right side of the heart or both sides of the heart.

Throughout the description, the terms “electric shock” and “pulse” are used interchangeably to refer to the electric current provided by a lead of an implantable heart device, whether it be a defibrillation shock or a pacing pulse. In addition, the term “lead” is used to describe both a standard pacemaker or ICD lead placed subcutaneously as well as a lead which forms part of a subcutaneous heart device as in the ISSD described below.

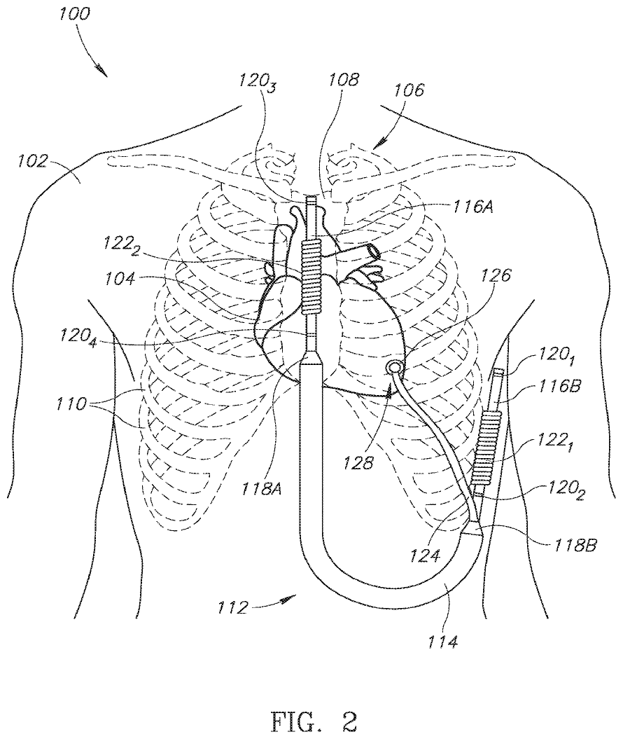

Reference is now made to FIG. 2, which is a schematic illustration of a subcutaneous implantable string shaped defibrillator with an epicardial lead positioned in a patient, generally referenced 100, constructed and operative in accordance with an embodiment of the disclosed technique. As mentioned above, the ISSD shown can be referred to as an ISSD-T. Shown in FIG. 2 is a human patient 102 with a subcutaneous ISSD-T 112 of the disclosed technique positioned around the heart. Human patient 102 is shown with a heart 104, a ribcage 106, a sternum 108 and plurality of ribs 110. Ribcage 106 protects heart 104. Subcutaneous ISSD-T 112 includes an elongated body 114, a first subcutaneous lead 116A and a second subcutaneous lead 116B and a first transition unit 118A and a second transition unit 118B. First subcutaneous lead 116A includes a plurality of sensing rings 1203 and 1204 as well as a defibrillation coil 1222. Second subcutaneous lead 116B includes a plurality of sensing rings 1201 and 1202 as well as a defibrillation coil 1221. First and second subcutaneous leads 116A and 116B are coupled with elongated body 114 via first and second transition units 118A and 118B respectively. Elongated body 114 may be flexible. Subcutaneous ISSD 112-T also includes an epicardial lead 124, coupled with elongated body 114 via second transition unit 118B. Epicardial lead 124 has an epicardial connector 126 for coupling an end of epicardial lead 124 to the apex of heart 104, as shown by an arrow 128.

As mentioned above, subcutaneous ISSD-T 112 is a unitary structure with no detachable parts and is completely subcutaneously implanted. As shown in FIG. 2, first subcutaneous lead 116A is positioned above sternum 108 whereas second subcutaneous lead 116B is positioned laterally outside of ribcage 106. Elongated body 114 is positioned mostly in the abdominal region of human patient 102. Epicardial lead 124 is threaded through plurality of ribs 110 such that epicardial connector 126 can be coupled with the apex of heart 104 for providing CRT, in particular to patients suffering from left bundle branch block. Epicardial connector 126 may include a shocking coil (not shown) for providing pacing and/or resynchronization shocks to heart 104. Epicardial connector 126 can be positioned over the left ventricle of the heart, as shown in FIG. 2.

Elongated body 114 includes a plurality of linked units or structures (not shown) which may include at least one battery, at least one capacitor for storing sufficient energy to provide at least one high voltage electric shock and a processor, for receiving metrics about the functioning of heart 104 and for determining the parameters of electric shocks delivered to heart 104. Elongated body 114 and/or first and second transition units 118A and 118B may also include at least one antenna (not shown) as well as at least one transmitter and receiver (both not shown), both coupled with the processor, for enabling a medical practitioner to program and communicate with subcutaneous ISSD-T 112 once it is implanted in a patient. The at least one battery may be either a non-rechargeable primary battery or a rechargeable battery. Plurality of sensing rings 1201-1204 is used to monitor metrics about the functioning of heart 104, such as the heart beat and various segments of the heart's electrical cycle and to pass the monitored metrics to the processor which can then decide if heart 104 is experiencing an arrhythmia and what kind of treatment via electric shocks should be provided. It is noted that at least one of the linked units may be an active segment such that a defibrillation vector can be applied from one of the defibrillation coils to the active segment.

Subcutaneous ISSD-T 112 as shown in FIG. 2 is substantially similar to the ISSD described in U.S. patent application Ser. No. 15/509,405, assigned to the same applicant as the current application, however that ISSD does not include an epicardial lead as shown in subcutaneous ISSD-T 112. Subcutaneous ISSD-T 112 may include a rechargeable power source, such as at least one rechargeable battery, which can be recharged used inductive recharging techniques. In the case where the ISSD-T uses a rechargeable battery, subcutaneous ISSD-T 112 can be recharged and therefore its physical size can be reduced as it does not need to store as much energy as an implantable ICD with a primary battery which cannot be recharged and must store sufficient energy for delivering electric shocks for the during of its operational life. In addition, in the case of using a rechargeable battery, due to its ability to recharge, the subcutaneous ISSD-T may have an operational life of 10-15 years or even longer. Furthermore, due to its subcutaneous placement, including the subcutaneous positioning of its subcutaneous leads, subcutaneous ISSD-T 112 can be implanted via minimally invasive surgical techniques, thus minimizing the general trauma caused by any surgery performed on a patient. In addition, the replacement of subcutaneous ISSD-T 112 after its operational life has ended is simplified as compared to prior art implantable ICDS as minimal invasive surgical techniques can be used to remove an old subcutaneous ISSD-T and implant a new subcutaneous ISSD-T.

Subcutaneous ISSD-T 112 of the disclosed technique is thus the equivalent of a subcutaneous CRT-D device with an epicardial lead however there is no need for any lead to be placed intravascularly in the heart of a patient in order to defibrillate the heart, if required. The epicardial lead enables the heart to be paced with elongated body 114 and its internal components functioning as an implantable pulse generator. The processor (not shown) in elongated body 114 can thus provide defibrillation shocks via defibrillation coils 1221 and 1222, pacing or resynchronization shocks via epicardial connector 126, or both, depending on the determined arrhythmia via plurality of sensing rings 1201-1204. The anticipative pacing algorithm for use with epicardial lead 124 is described below.

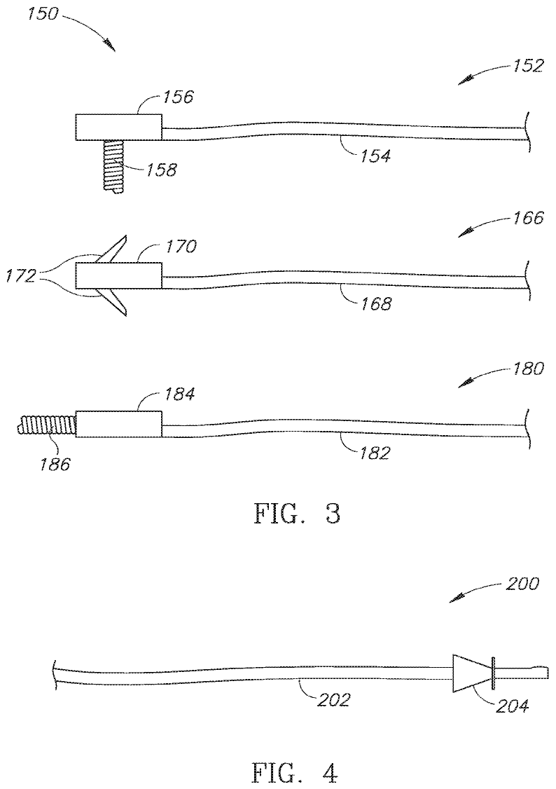

According to the disclosed technique, the epicardial lead can be implanted via a plethora of known techniques to access the pericardial space. One such technique was already described in the background section. In another embodiment of the disclosed technique, the implantation procedure of the epicardial lead is performed by first making an approximately 2 centimeter incision in the lateral thorax and via this mini-thoracotomy incising the pericardium. Next, taking care not to damage a coronary artery or vein, the epicardial lead is sewn to the lateral region of the left ventricle typically between the apex and base of the left ventricle. As shown below in FIG. 3, a typical lead used in the disclosed technique as an epicardial lead has a sewing collar to allow for attachment of the lead to the ventricle and a cork-screw type electrode which is screwed into the myocardium thus coupling the epicardial lead with the outer surface of the heart.

Reference is now made to FIG. 3, which is a schematic illustration of various epicardial lead ends for coupling the epicardial lead to a left ventricle of a heart, generally referenced 150, constructed and operative in accordance with another embodiment of the disclosed technique. Shown are a first epicardial lead 152, a second epicardial lead 166 and a third epicardial lead 180. Each one of epicardial leads 152, 166 and 180 are substantially similar to epicardial lead 124 (FIG. 2) and represent various embodiments for embodying the epicardial lead of the disclosed technique. First epicardial lead 152 includes a lead body 154 which includes a lead wire (not shown) and a biocompatible coating (not shown). Lead body 154 is terminated by an epicardial connector 156 for coupling first epicardial lead 152 to the outer surface of a heart (not shown). Epicardial connector 156 includes a vertical screw hook 158 which can be used to couple epicardial connector 156 to the outer surface of the heart. Second epicardial lead 166 includes a lead body 168 which includes a lead wire (not shown) and a biocompatible coating (not shown). Lead body 168 is terminated by an epicardial connector 170 for coupling second epicardial lead 166 to the outer surface of the heart. Epicardial connector 170 includes a plurality of anchor wings 172 which can be used to couple epicardial connector 170 to the outer surface of the heart. Third epicardial lead 180 includes a lead body 182 which includes a lead wire (not shown) and a biocompatible coating (not shown). Lead body 182 is terminated by an epicardial connector 184 for coupling third epicardial lead 180 to the outer surface of the heart. Epicardial connector 184 includes a horizontal screw hook 186 which can be used to couple epicardial connector 184 to the outer surface of the heart. The epicardial leads and epicardial connectors shown in FIG. 3 are examples of epicardial connectors for use with the disclosed technique. Other epicardial connectors known in the art can also be used to couple the epicardial lead of the disclosed technique to the outer surface of the heart, such as those made as patches. In addition, the epicardial lead ends shown and described can be used to couple an epicardial lead to a right side (e.g., right ventricle or right atrium) of the heart as well as a left side (e.g., left ventricle or left atrium) of the heart.

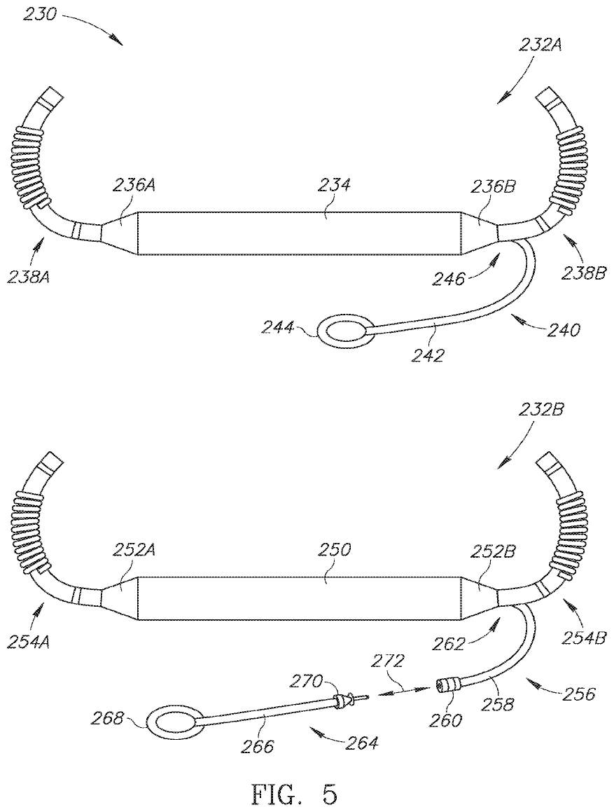

Reference is now made to FIG. 4, which is a schematic illustration of an epicardial lead end for coupling the epicardial lead to a subcutaneous implantable string shaped defibrillator, generally referenced 200, constructed and operative in accordance with a further embodiment of the disclosed technique. Shown is a lead body 202 and a lead end 204. Lead body 202 may be any of lead bodies 154, 168 or 182 (all in FIG. 3). Lead end 204 may be a standard IS-1 connector. As described below in FIG. 5, in one embodiment of the disclosed technique, the epicardial lead may be embodied as a standard epicardial lead with a standard IS-1 connector, or standard IS-1 bipolar connector and is thus attachable and detachable from the subcutaneous ISSD-T. According to this embodiment of the disclosed technique, the epicardial lead can be attached to any standard IPG. In another embodiment of the disclosed technique, the epicardial lead may be permanently attached to the elongated body of the subcutaneous ISSD-T like the subcutaneous leads described below in FIG. 5 and is thus not detachable. In either embodiment, the epicardial lead is inserted subcutaneously via a surgical incision made near the apex of the heart of the patient and the epicardial connector is then coupled with the outer surface of the heart either using a screw hook, anchor wings (as shown above in FIG. 3), a patch or other mechanical fastening mechanisms. In addition, the epicardial connector may be coupled to the apex via sutures or a suturing structure as is known in the medical field.

Reference is now made to FIG. 5, which is a schematic illustration of variations of the subcutaneous implantable string shaped defibrillator with an epicardial lead of FIG. 2, generally referenced 230, constructed and operative in accordance with another embodiment of the disclosed technique. FIG. 5 shows two variations of the subcutaneous ISSD of the disclosed technique with an epicardial lead as a first subcutaneous ISSD-T 232A and a second subcutaneous ISSD-T 232B. First subcutaneous ISSD-T 232A includes an elongated body 234, two subcutaneous leads 238A and 238B, two transition units 236A and 236B and an epicardial lead 240. Subcutaneous leads 238A and 238B and epicardial lead 240 are coupled with elongated body 234 via transition units 236A and 236B. Elongated body 234 may be flexible. Epicardial lead 240 is shown being permanently coupled with transition unit 236B via an arrow 246 however this is merely an example and epicardial lead 240 could instead be permanently coupled with transition unit 236A (not shown). Epicardial lead 240 is thus integrated with elongated body 234 via transition unit 236B. Subcutaneous leads 238A and 238B each include a defibrillation coil (shown but not labeled) and two sensing rings (also shown but not labeled). Epicardial lead 240 is shown having a lead body 242 and an epicardial connector 244. Lead 242 and epicardial connector 244 could be any of the lead bodies and epicardial connectors shown and described above in FIG. 3.

Elongated body 234 includes a plurality of units which encapsulate at least one battery (either rechargeable or non-rechargeable), at least one capacitor and a processor (all not shown) as well as electrical connections between these components and the subcutaneous leads 238A and 238B and epicardial lead 240. First subcutaneous ISSD-T 232A is completely unitary and forms a single device with no detachable parts. The placement of first subcutaneous ISSD-T 232A in the body of a patient was shown above in FIG. 2.

Second subcutaneous ISSD 232B-T includes an elongated body 250, two subcutaneous leads 254A and 254B, two transition units 252A and 252B and a female epicardial lead 256. Elongated body 250 may be flexible. Subcutaneous leads 254A and 254B and female epicardial lead 256 are coupled with elongated body 250 via transition units 252A and 252B. Female epicardial lead 256 is shown being coupled with transition unit 252B via an arrow 262 however this is merely an example and female epicardial lead 256 could instead be coupled with transition unit 252A (not shown). Female epicardial lead 256 includes a lead body 258 and a female connector 260. Female connector 260 may be a standard female IS-1 connector. Female connector 260 can also be other types of connectors as known in the art. Shown as well is a male epicardial lead 264 which includes a lead body 266, a male connector 270 and an epicardial connector 268. Male connector 270 can be a standard male IS-1 connector and can be coupled with female connector 260 as shown by an arrow 272. Male connector 270 can also be other types of connectors as known in the art. Lead body 266 and epicardial connector 268 can be any of the lead bodies and epicardial connectors shown above in FIG. 3 and male connector 270 could be the lead end shown above in FIG. 4. Second subcutaneous ISSD-T 232B is thus a single device with a detachable epicardial lead. Female epicardial lead 256 is integrated with elongated body 250 via transition unit 252B.

Subcutaneous leads 254A and 254B each include a defibrillation coil (shown but not labeled) and two sensing rings (also shown but not labeled). Elongated body 250 includes a plurality of units which encapsulate at least one battery (either rechargeable or non-rechargeable), at least one capacitor and a processor (all not shown) as well as electrical connections between these components and the subcutaneous leads 254A and 254B and female epicardial lead 256. In this embodiment, male epicardial lead 264 can be embodied as any off-the-shelf epicardial lead using the standard IS-1 connector such as the MYODEX™ epicardial pacing lead from ST. JUDE MEDICAL™ and the CAPSURE EPI® models 10366 and 4968 epi/myocardial pacing leads from Medtronic. Male epicardial lead 264 can also be other epicardial leads having a connector.

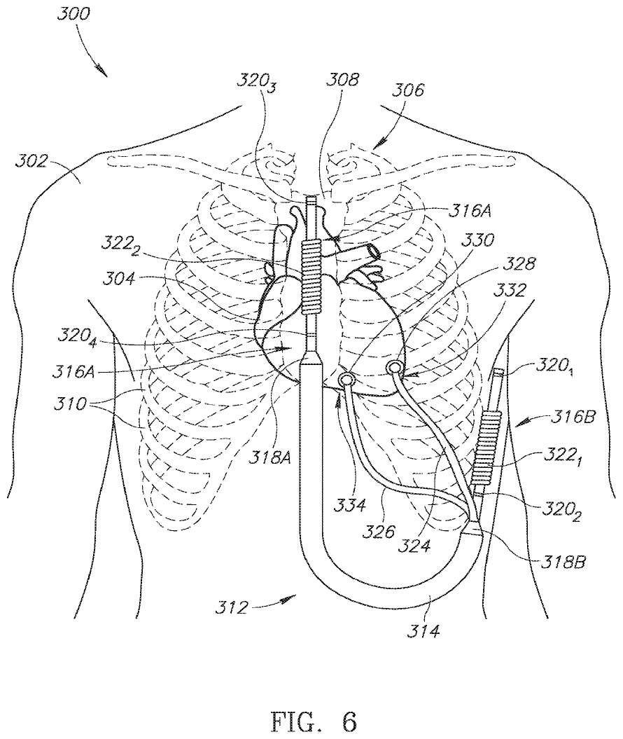

Reference is now made to FIG. 6, which is a schematic illustration of a subcutaneous implantable string shaped defibrillator with two epicardial leads, generally referenced 300, constructed and operative in accordance with a further embodiment of the disclosed technique. Shown in FIG. 6 is a human patient 302 with a subcutaneous ISSD-T 312 of the disclosed technique positioned around the heart. Human patient 302 is shown with a heart 304, a ribcage 306, a sternum 308 and a plurality of ribs 310. Ribcage 306 protects heart 304. Subcutaneous ISSD-T 312 includes an elongated body 314, a first subcutaneous lead 316A and a second subcutaneous lead 316B and a first transition unit 318A and a second transition unit 318B. First subcutaneous lead 316A includes a plurality of sensing rings 3203 and 3204 as well as a defibrillation coil 3222. Second subcutaneous lead 316B includes a plurality of sensing rings 3201 and 3202 as well as a defibrillation coil 3221. First and second subcutaneous leads 316A and 316B are coupled with elongated body 314 via first and second transition units 318A and 318B respectively. Subcutaneous ISSD-T 312 also includes a first epicardial lead 324 and a second epicardial lead 326, both coupled with elongated body 314 via second transition unit 318B. First and second epicardial leads 324 and 326 could also be coupled with elongated body 314 via first transition unit 318A. First epicardial lead 324 has an epicardial connector 328 and second epicardial lead 326 has an epicardial connector 330. Epicardial connector 328 couples first epicardial lead 324 to the left side of heart 304, as shown by an arrow 332, and epicardial connector 330 couples second epicardial lead 326 to the right side of heart 304, as shown by an arrow 334. First epicardial lead 324 and second epicardial lead 326 can be positioned on other areas of heart 304 (not shown), such as the apex of the heart (not labeled), the right and/or left atria of the heart (not labeled) or on other sections of the right and/or left ventricles (not shown).

Subcutaneous ISSD-T 312 is substantially similar to subcutaneous ISSD-T 112 (FIG. 2) except that it includes two epicardial leads instead of one. Having two epicardial leads, subcutaneous ISSD-T 312 can be used to deliver electric shocks to the left ventricle via first epicardial lead 324 and to the right ventricle via second epicardial lead 326, thereby providing bi-ventricular pacing to heart 304. First epicardial lead 324 can also be used to synchronize the left side of heart 304 in cases of left bundle branch block. Second epicardial lead 326 can also be used for standard pacing of the right ventricle such as for the treatment of bradycardia. Thus subcutaneous ISSD-T 312 can function as a cardioversion defibrillator via first and second subcutaneous leads 316A and 316B and also as a pacemaker for pacing both ventricles of heart 304 via first and second epicardial leads 324 and 326. According to the disclosed technique, with two epicardial leads coupled with the heart as well as two subcutaneous leads positioned around the heart, pacing of the patient's heart (in particular the ventricles) can be individualized to a patient's particular cardiac physiology. For example, in certain patients, it might be advantageous to pace only the left ventricle using the anticipative pacing method of the disclosed technique. In some patients, simultaneous pacing of both the right and left ventricles can be performed, while in other patients, only right ventricle pacing should be delivered. Still in other patients, both right ventricle pacing and left ventricle pacing can be delivered with the timing of the respective right ventricle and left ventricle pacing impulses individualized to benefit the particular patient's cardiac physiology. For example, the anticipative pacing method of the disclosed technique can be used to anticipate right ventricle conduction and used to pace the left ventricle prior to right bundle branch conduction since in many instances this can maximize synchronization between the two ventricles in circumstances where bi-ventricular pacing is not wanted. As another example, there might be advantages to left ventricle only pacing wherein anticipative left ventricle pacing delivery using the anticipative pacing method mentioned above would allow for native right bundle branch conduction, which could provide a more potentially physiologic right ventricular contraction as the native conduction system would be used in this circumstance. It is noted as well that pacing only the left ventricle via the anticipative pacing method of the disclosed technique will decrease the overall energy requirement needed for CRT type pacing.

As shown in FIG. 6, subcutaneous ISSD-T 312 is embodied as a unitary structure with no detachable parts and is completely subcutaneously implanted. First subcutaneous lead 316A is positioned above sternum 308 whereas second subcutaneous lead 316B is positioned laterally outside of ribcage 306. Elongated body 314 is positioned mostly in the abdominal region of human patient 302. First and second epicardial leads 324 and 326 are threaded through plurality of ribs 310 such that epicardial connectors 328 and 330 can be coupled with the ventricles of heart 304 for providing CRT as well as bi-ventricular pacing, in particular to patients suffering from left BBB. Epicardial connectors 328 and 330 may each include a shocking coil (not shown) for providing pacing and/or resynchronization shocks to heart 304. It is noted that each of first and second epicardial leads 324 and 326 can be embodied either as epicardial lead 240 (FIG. 5), i.e. as an integrated lead or as female epicardial lead 256 (FIG. 5) and male epicardial lead 264 (FIG. 5), i.e. a detachable lead.

Elongated body 314 includes a plurality of linked units or structures (not shown) which may include at least one battery (either rechargeable or non-rechargeable), at least one capacitor for storing sufficient energy to provide at least one high voltage electric shock and a processor, for receiving metrics about the functioning of heart 304 and for determining the parameters of electric shocks delivered to heart 304. Plurality of sensing rings 3201-3204 is used to monitor metrics about the functioning of heart 304 and to pass the monitored metrics to the processor which can then decide if heart 304 is experiencing an arrhythmia and what kind of treatment via electric shocks should be provided.

As mentioned above, subcutaneous ISSD-T 312 as shown in FIG. 6 is substantially similar to the ISSD described in U.S. patent application Ser. No. 15/509,405, assigned to the same applicant as the current application, however that ISSD does not include one or more epicardial leads. Subcutaneous ISSD-T 312 includes a power source, such as at least one battery (either rechargeable or non-rechargeable), which can be recharged used inductive recharging techniques in the case of the battery being rechargeable. In the rechargeable case, since subcutaneous ISSD-T 312 can be recharged, its physical size can be reduced as it does not need to store as much energy as an implantable ICD which cannot be recharged and must store sufficient energy for delivering electric shocks for the duration of its operational life. In addition, due to its ability to recharge, such a subcutaneous ISSD-T may have an operational life of 10-15 years. Furthermore, due to its subcutaneous placement, including the subcutaneous positioning of its subcutaneous leads, subcutaneous ISSD-T 312 can be implanted via minimally invasive surgical techniques, thus minimizing the general trauma caused by any surgery performed on a patient. In addition, the replacement of subcutaneous ISSD-T 312 after its operational life has ended is simplified as compared to prior art implantable ICDS as minimal invasive surgical techniques can be used to remove an old subcutaneous ISSD-T and implant a new subcutaneous ISSD-T.

It is noted that both subcutaneous ISSD-T 112 (FIG. 2) and subcutaneous ISSD-T 312 can apply various kinds of electric shock therapies to the heart of a human patient. As mentioned above, an issue with prior art subcutaneous ICDs is that in order to provide CRT, the ICD needs to know when to pace the ventricles, which is usually determined by an intravascular lead placed in or near the right and/or left ventricles. This could also be achieved using a pill pacemaker placed inside one of the ventricles. According to the disclosed technique, the determination of when to pace the right and/or left ventricles can be performed by using an anticipative pacing method for determining when each ventricle should be paced. The anticipative pacing method of the disclosed technique can be used by the processor in the elongated body of subcutaneous ISSD-T 112 or subcutaneous ISSD-T 312 to determine when pacing pulses should be sent via at least one of the epicardial leads coupled to the outer surface of the heart.

The anticipative pacing method of the disclosed technique works as follows. The sensors of the subcutaneous heart device of the disclosed technique (such as a subcutaneous ISSD-T or a subcutaneous IPG with a subcutaneously or substernally placed lead and an epicardial lead) are used to build a database of the electrical impulses of a patient, for example one suffering from BBB. Recorded values in the database may include the time duration of a P-wave, a QRS complex and a T-wave for every P-P interval. The time delay of the intrinsically conducted atrioventricular (herein abbreviated AV) interval (i.e., the P-R segment) for various different heart rates is also incorporated into the database. Based on the database, the subcutaneous heart device can anticipate the AV delay of a given P-P interval and deliver an electrical impulse to cause a ventricle suffering from BBB to contract in sync with the other ventricle. P-P intervals change in time duration as the heart rate varies from moment to moment. The AV interval (i.e., the AV delay) for any given heart rate also varies but in a given individual patient, at a given P-P interval, the AV interval tends to remain relatively constant from day to day for that given P-P interval. Using the built database, the AV delay used to anticipate when a ventricle is to be paced is changed dynamically, according to the disclosed technique, to match the native pumping of the heart (based on the patient's current heart rate), thus optimizing the cardiac cycle of a patient suffering from BBB. According to the method of the disclosed technique, the database is periodically verified and modified if the recorded value of a wave, complex or segment (e.g. P-wave, QRS complex, T-wave and the like) for a particular time duration in a given P-P interval changes.

The P-R segment in an electrocardiogram (herein abbreviated ECG), which represents the AV delay in the heart, includes three sub-delays known as the intra-atrial conduction time, the AV nodal conduction time and the infra-Hisian conduction time. According to the disclosed technique, an analysis of the AV delay in humans with normal and abnormal heart physiologies shows that intra-atrial conduction time and infra-Hisian conduction time are largely constant and fixed and do not significantly vary from day to day or from moment to moment (typical inter-atrial conduction time is 5-10 ms and typical infra-Hisian conduction time is 40-55 ms). However, AV nodal conduction time varies according to inputs the heart receives from the autonomic nervous system (herein abbreviated ANS). These same inputs from the ANS also control the automaticity of the sinoatrial (herein abbreviated SA) node and thus influence the time duration of the P-P interval in the cardiac cycle. AV nodal conduction times can thus vary greatly, for example between 70-300 ms, depending on the time of day and the moment a person finds oneself in. However, since AV nodal conduction times are controlled by the same inputs the SA node receives, the variations in AV nodal conduction times are substantially related to the time duration of the P-P interval, i.e., the heart rate. According to the disclosed technique, during periods of slower heart rates, the AV nodal conduction times will tend to be longer and vice-versa, during periods of rapid heart rates, the AV nodal conduction times will be shorter. For a given individual it is thus possible to measure AV nodal conduction times at various heart rates and build a database of AV nodal conduction times for any given heart rate. This database can then be used to predict and anticipate what the AV nodal conduction time will be for any given individual at any given heart rate and forms the basis for the anticipative pacing method and algorithm described above.

In particular, both subcutaneous ISSD-Ts 112 (FIGS. 2) and 312 can apply a dynamic anticipative pacing algorithm and method as described above and as described in more detail in U.S. Pat. No. 9,352,159, assigned to the same applicant as the current application, via the epicardial lead or leads. It is noted as well that subcutaneous ISSD-T 312 is shown having two epicardial leads, however according to the disclosed technique, a plurality of epicardial leads can be included in subcutaneous ISSD-T 312, coupled with elongated body 314 via at least one of first and second transition units 318A and 318B. It is furthermore noted that according to the disclosed technique, the epicardial lead or leads coupled with the heart can be used to determine when to pace the left and/or right ventricle by direct measurement from the surface of the heart. In such an embodiment, the epicardial connector includes a sensor (not shown) for recording and determining the electrical activity of a ventricle.

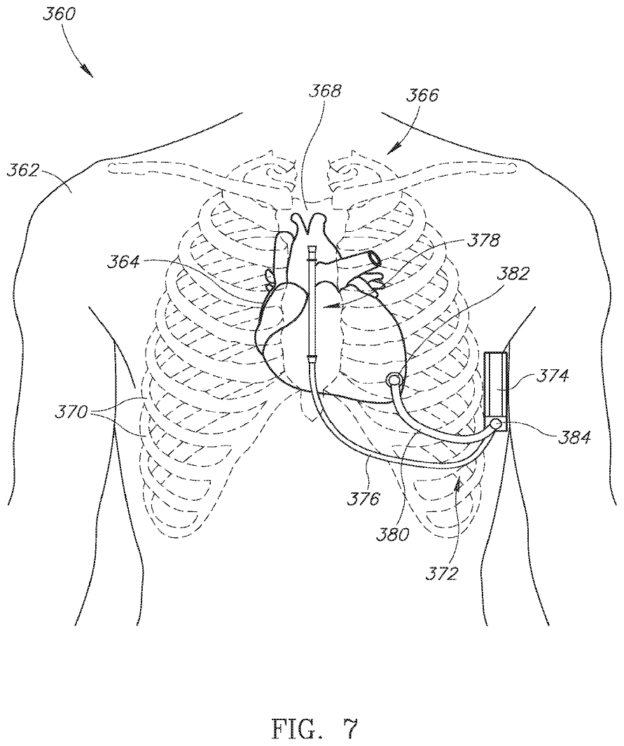

Reference is now made to FIG. 7, which is a schematic illustration of a subcutaneous ICD with an epicardial lead, generally referenced 360, constructed and operative in accordance with another embodiment of the disclosed technique. Shown in FIG. 7 is a human patient 362 with a subcutaneous ICD 372 of the disclosed technique positioned around the heart. Human patient 362 is shown with a heart 364, a ribcage 366, a sternum 368 and plurality of ribs 370. Ribcage 366 protects heart 364. Subcutaneous ICD 372 includes an IPG 374, a subcutaneous lead 376, an epicardial lead 380 and a connector box 384. Subcutaneous lead 376 includes a defibrillator lead end 378 which includes a defibrillating coil (not shown) and a plurality of sensors (not shown). Epicardial lead 380 includes an epicardial connector 382 for coupling epicardial lead 380 to heart 364. Both subcutaneous lead 376 and epicardial lead 380 are coupled with IPG 374 via connector box 384. IPG 374 may include an active section which is electrically active. Epicardial lead 380 is shown coupled with the left ventricle of heart 364 whereas subcutaneous lead 376 is positioned over sternum 368. It is noted that subcutaneous lead 376 can be positioned under sternum 368 (i.e. substernally) or at different positions vis-à-vis sternum 368 (such as to the left or right of the sternum—not shown). Epicardial lead 380 can be positioned on other areas of heart 364 (not shown), such as the apex of the heart (not labeled), the right and/or left atria of the heart (not labeled) or on other sections of the right and/or left ventricles (not shown).

As shown in FIG. 7, subcutaneous ICD 372 can be used as a defibrillator via subcutaneous lead 376 and IPG 374 (which may function as a shocking coil if IPG 374 includes an electrically active segment) and also as a CRT device via epicardial lead 380 using the anticipative pacing method and algorithm described above. Epicardial lead 380 can be embodied either as epicardial lead 240 (FIG. 5), i.e. an integrated lead or as female epicardial lead 256 (FIG. 5) and male epicardial lead 264 (FIG. 5), i.e. a detachable lead.

As mentioned above, the disclosed technique has been described using the example of an ISSD-T (FIGS. 2, 5 and 6) and a subcutaneous ICD with an epicardial lead (FIG. 7), however the disclosed technique is not limited to such heart devices. The disclosed technique can be embodied by any subcutaneously implanted heart device which can function as a subcutaneous defibrillator without having any leads in the heart or in the vascular system. It is noted as well that the epicardial lead of the disclosed technique can be used to determine when to pace the left and/or right ventricle of the heart due to its coupling with the outer surface of the heart and thus an anticipative pacing algorithm or method may not be necessary to determine when the left and/or right ventricles should be paced.

It will be appreciated by persons skilled in the art that the disclosed technique is not limited to what has been particularly shown and described hereinabove. Rather the scope of the disclosed technique is defined only by the claims, which follow.

1. A method, comprising:
a. providing a subcutaneous implantable string shaped defibrillator (ISSD-T) comprising: a flexible elongated body, at least two defibrillation leads for providing at least one cardioversion defibrillation shock, at least one sensor positioned on at least one of the at least two defibrillation leads configured to determine at least one metric of a heart, at least one epicardial lead coupled with the flexible elongated body via at least one of the at least two transition units for providing at least one cardiac resynchronization therapy (CRT) pulse; and
b. implanting the at least two defibrillation leads and at least two transition units in a superficial subcutaneous positioning under the skin of a patient,
wherein the flexible elongated body comprises a plurality of linked units, wherein at least a first one of the plurality of linked units encapsulates at least one capacitor,
wherein at least a second one of the plurality of linked units encapsulates at least one power source,
wherein at least a third one of the plurality of linked units encapsulates a processor, and
wherein the processor provides at least one signal to the at least one epicardial lead for providing the at least one CRT pulse.

2. The method of claim 1, further comprising coupling the at least one epicardial lead to an outer surface of the heart of the patient.

3. The method of claim 1, wherein the at least one epicardial lead is permanently coupled with the at least one of the at least two transition units.

4. The method of claim 1, wherein the at least one epicardial lead comprises a male end and a female end, wherein the female end is permanently coupled with the at least one of the at least two transition units and wherein the male end is detachable from the female end.

5. The method of claim 4, wherein the male end comprises a connector.

6. The method of claim 1, further comprising coupling a first one of the at least one epicardial leads to the left of an apex of the heart for left ventricle pacing and coupling a second one of the at least one epicardial leads to the right of the apex of the heart for right ventricle pacing.

7. The method of claim 1, wherein the at least one epicardial lead includes: a lead body, and an epicardial connector coupled to a distal end of the lead body for coupling the at least one epicardial lead with an outer surface of the heart.

8. The method of claim 7, wherein the epicardial connector includes at least one of: a vertical screw hook, a plurality of anchor wings, and a horizontal screw hook.

9. The method of claim 1, wherein the processor provides the at least one signal to the at least one epicardial lead using an anticipative pacing algorithm.

10. The method of claim 1, wherein the processor provides at least one signal to the at least one epicardial lead based on a coupling of the at least one epicardial lead to an outer surface of the heart.

11. The method of claim 1, wherein at least one of the plurality of linked units is an active segment and wherein the at least one cardioversion defibrillation shock is applied between at least one of the at least two defibrillation leads and the active segment.

12. The method of claim 1, wherein the power source includes at least one rechargeable battery and wherein the method further comprises recharging the at least one rechargeable battery using inductive recharging.

13. A method comprising:
a. providing a subcutaneous implantable defibrillator for providing cardiac resynchronization therapy (CRT), the subcutaneous implantable defibrillator comprising: an implantable pulse generator (IPG), at least one defibrillation lead, and at least one epicardial lead for providing at least one CRT pulse; and
b. implanting the IPG and at least one defibrillation lead in a superficial subcutaneous position under the skin of a patient,
wherein the IPG comprises: a connector box for coupling the at least one defibrillation lead and the at least one epicardial lead with the IPG, at least one capacitor, at least one power source, and at least one electronic circuit,
wherein the at least one defibrillation lead comprises at least one sensor for determining at least one metric of the heart, and
wherein the at least one epicardial lead is configured to provide the at least one CRT pulse according to the at least one determined metric.

14. The method of claim 13, further comprising coupling the at least one epicardial lead to an outer surface of the heart of the patient.

15. The method of claim 13, where the at least one epicardial lead is permanently coupled with the IPG.

16. The method of claim 13, wherein the at least one epicardial lead comprises a male end and a female end, wherein the female end is permanently coupled with the connector box and wherein the male end is detachable from the female end.

17. The method of claim 16, wherein the male end comprises a connector.

18. The method of claim 13, wherein the at least one defibrillation lead is positioned subcutaneously near the heart.

19. The method of claim 13, wherein the at least one defibrillation lead is positioned substernally near the heart.

20. The method of claim 13, further comprising coupling a first one of the at least one epicardial leads to the left of an apex of the heart for left ventricle pacing and coupling a second one of the at least one epicardial leads to the right of the apex of the heart for right ventricle pacing.

21. The method of claim 13, wherein the at least one epicardial lead includes a lead body, and an epicardial connector coupled to a distal end of the lead body, for coupling the at least one epicardial lead with an outer surface of the heart.

22. The method of claim 21, wherein the epicardial connector comprises at least one of:
a vertical screw hook, a plurality of anchor wings, and a horizontal screw hook.

23. The method of claim 13, wherein the at least one electronic circuit provides at least one signal to the at least one epicardial lead to provide the at least one CRT pulse using an anticipative pacing algorithm.

24. The method of claim 13, wherein the at least one electronic circuit provides at least one signal to the at least one epicardial lead to provide the at least one CRT pulse based on a coupling of the at least one epicardial lead to an outer surface of the heart.

25. The method of claim 13, wherein the IPG comprises an electrically active section and wherein the at least one CRT pulse is applied between the at least one defibrillation lead and the electrically active section of the IPG.

26. The method of claim 13, wherein the power source includes at least one rechargeable battery and wherein the method further comprises recharging the at least one rechargeable battery using inductive recharging.
